(19) United States
(12) Patent Application Publication
(10) Pub. No.: US 2005/0085717 A1
(43) Pub. Date: Apr. 21, 2005

(54) Systems and methods for intraoperative targetting

(76) Inventor: Ramin Shahidi, Palo Alto, CA (US)

Correspondence Address: STATTLER, JOHANSEN, AND ADELI LLP, 1875 CENTURY PARK EAST SUITE 1050, CENTURY CITY, CA, 90067, US

(21) Appl. No.: 10/764,650

(22) Filed: Jan. 26, 2004

Related U.S. Application Data
(60) Provisional application No. 60/513,157, filed on Oct. 21, 2003.

(51) Int. Cl. A61B 1/04; A61B 8/12; A61B 8/14

(52) U.S. Cl. 600/424; 128/922; 600/443; 600/109; 600/117

(57) ABSTRACT

Systems and methods are disclosed for assisting a user in guiding a medical instrument to a subsurface target site in a patient by indicating a spatial feature of a patient target site on an intraoperative image (e.g., endoscopic image), determining 3-D coordinates of the patient target site spatial feature in a reference coordinate system using the spatial feature of the target site indicated on the intraoperative image (e.g., ultrasound image, determining a position of the instrument in the reference coordinate system, projecting onto a display device a view field from a predetermined position relative to the instrument in the reference coordinate system, and projecting onto the view field an indicia of the spatial feature of the target site corresponding to the predetermined position.

This Application claims priority from Provisional Application Ser. No. 60/513,157 filed on Oct. 21, 2003 and entitled “SYSTEMS AND METHODS FOR SURGICAL NAVIGATION”, the content of which is incorporated by referenced herewith.

### BACKGROUND

In recent years, the medical community has been increasingly focused on minimizing the invasiveness of surgical procedures. Advances in imaging technology and instrumentation have enabled procedures using minimally-invasive surgery with very small incisions. Growth in this category is being driven by a reduction in morbidity relative to traditional open procedures, because the smaller incisions minimize damage to healthy tissue, reduce patient pain, and speed patient recovery. The introduction of miniature CCD cameras and their associated microelectronics has broadened the application of endoscopy from an occasional biopsy to full minimally-invasive surgical ablation and aspiration.

Minimally-invasive endoscopic surgery offers advantages of a reduced likelihood of intraoperative and post-operative complications, less pain, and faster patient recovery. However, the small field of view, the lack of orientation cues, and the presence of blood and obscuring tissues combine to make video endoscopic procedures in general disorienting and challenging to perform. Modern volumetric surgical navigation techniques have promised better exposure and orientation for minimally-invasive procedures, but the effective use of current surgical navigation techniques for soft tissue endoscopy is still hampered by compensating for tissue deformations and target movements during an interventional procedure.

To illustrate, when using an endoscope, the surgeon's vision is limited to the camera's narrow field of view and the lens is often obstructed by blood or fog, resulting in the surgeon suffering a loss of orientation. Moreover, endoscopes can display only visible surfaces and it is therefore often difficult to visualize tumors, vessels, and other anatomical structures that lie beneath opaque tissue (e.g., targeting of pancreatic adenocarcinomas via gastro-intestinal endoscopy, or targeting of submucosal lesions to sample peri-intestinal structures such as masses in the liver, or targeting of subluminal lesion in the bronchi).

Recently, image-guided therapy (IGT) systems have been introduced. These systems complement conventional endoscopy and have been used predominantly in neurological, sinus, and spinal surgery, where bony or marker-based registration can provide adequate target accuracy using pre-operative images (typically 1-3 mm). While IGT enhances the surgeon's ability to direct instruments and target specific anatomical structures, in soft tissue these systems lack sufficient targeting accuracy due to intra-operative tissue movement and deformation. In addition, since an endoscope provides a video representation of a 3D environment, it is difficult to correlate the conventional, purely 2D IGT images with the endoscope video. Correlation of information obtained from intra-operative 3D ultrasonic imaging with video endoscopy can significantly improve the accuracy of localization and targeting in minimally-invasive IGT procedures.

Until the mid 1990's, the most common use of image guidance was for stereotactic biopsies, in which a surgical trajectory device and a frame of reference were used. Traditional frame-based methods of stereotaxis defined the intracranial anatomy with reference to a set of fiducial markers, which were attached to a frame that was screwed into the patient's skull. These fiducials were measured on pre-operative tomographic (MRI or CT) images.

A trajectory-enforcement device was placed on top of the frame of reference and used to guide the biopsy tool to the target lesion, based on prior calculations obtained from pre-operative data. The use of a mechanical frame allowed for high localization accuracy, but caused patient discomfort, limited surgical flexibility, and did not allow the surgeon to visualize the approach of the biopsy tool to the lesion. There has been a gradual emergence of image guided techniques that eliminate the need for the frame altogether. The first frameless stereotactic system used an articulated robotic arm to register pre-operative imaging with the patient's anatomy in the operating room. This was followed by the use of acoustic devices for tracking instruments in the operating environment. The acoustic devices eventually were superceded by optical tracking systems, which use a camera and infrared diodes (or reflectors) attached to a moving object to accurately track its position and orientation. These systems use markers placed externally on the patient to register pre-operative imaging with the patient's anatomy in the operating room. Such intra-operative navigation techniques use pre-operative CT or MR images to provide localized information during surgery. In addition, all systems enhance intra-operative localization by providing feedback regarding the location of the surgical instruments with respect to 2D preoperative data.

Today, surgical navigation systems are able to provide real-time fusion of pre-operative 3D data with intraoperative 2D data images such as endoscopes. These systems have been used predominantly in neurological, sinus, and spinal surgery, where direct access to the pre-operative data plays a major role in the execution of the surgical task. The novelty of the techniques and methods set forth here are in the capability of providing navigational and targeting information from any perspective, only using intraoperative images; thus eliminating the need for the use of preoperative images all together.

### SUMMARY

In one aspect, a method for assisting a user in guiding a medical instrument to a subsurface target site in a patient includes generating one or more intraoperative images on which a spatial feature of a patient target site can be indicated, indicating a spatial feature of the target site on said image(s), using the spatial feature of the target site indicated on said image(s) to determine 3-D coordinates of the target site spatial feature in a reference coordinate system, tracking the position of the instrument in the reference coordinate system, projecting onto a display device, a view field as seen from a known position and, optionally, a known orientation, with respect to the tool, in the reference coordinate system, and projecting onto the displayed view field, indicia whose states are related to the indicated spatial feature of the target site with respect to said known position and, optionally, said known orientation, whereby the user, by observing the states of said indicia, can guide the instrument toward the target site by moving the instrument so that said indicia are placed or held in a given state in the displayed field of view.

The generating includes using an ultrasonic source to generate an ultrasonic image of the patient, and the 3-D coordinates of a spatial feature indicated on said image are determined from the 2-D coordinates of the spatial feature on the image and the position of the ultrasonic source. The medical instrument can be an endoscope and the view field projected onto the display device can be the image seen by the endoscope. The view field projected onto the display device can be that seen from the tip-end position and orientation of the medical instrument having a defined field of view. The view field projected onto the display device can be that seen from a position along the axis of instrument that is different from the tip-end position of the medical instrument. The target site spatial feature indicated can be a volume or area, and said indicia are arranged in a geometric pattern which defines the boundary of the indicated spatial feature. The target site spatial feature indicated can be a volume, area or point, and said indicia are arranged in a geometric pattern that indicates the position of a point within the target site. The spacing between or among indicia can be indicative of the distance of the instrument from the target-site position. The size or shape of the individual indicia can indicate the distance of the instrument from the target-site position. The size or shape of individual indicia can also be indicative of the orientation of said tool. The indicating includes indicating on each image, a second spatial feature which, together with the first-indicated spatial feature, defines a surgical trajectory on the displayed image. The instrument can indicate on a patient surface region, an entry point that defines, with said indicated spatial feature, a surgical trajectory on the displayed image. The surgical trajectory on the displayed image can be indicated by two sets of indicia, one set corresponding to the first-indicated spatial feature and the second, by the second spatial feature or entry point indicated. The surgical trajectory on the displayed image can be indicated by a geometric object defined, at its end regions, by the first-indicated spatial feature and the second spatial feature or entry point indicated.

In another aspect, a system for guiding a medical instrument to a target site in a patient includes an imaging device for generating one or more intraoperative images, on which spatial features of a patient target site can be defined in a 3-dimensional coordinate system, a tracking system for tracking the position and optionally, the orientation of the medical instrument and imaging device in a reference coordinate system, an indicator by which a user can indicate a spatial feature of a target site on such image(s), a display device, an electronic computer operably connected to said tracking system, display device, and indicator, and computer-readable code which is operable, when used to control the operation of the computer, to perform (i) recording target-site spatial information indicated by the user on said image(s), through the use of said indicator, (ii) determining from the spatial feature of the target site indicated on said image(s), 3-D coordinates of the target-site spatial feature in a reference coordinate system, (iii) tracking the position of the instrument in the reference coordinate system, (iv) projecting onto a display device, a view field as seen from a known position and, optionally, a known orientation, with respect to the tool, in the reference coordinate system, and (v) projecting onto the displayed view field, indicia whose states indicate the indicated spatial feature of the target site with respect to said known position and, optionally, said known orientation, whereby the user, by observing the states of said indicia, can guide the instrument toward the target site by moving the instrument so that said indicia are placed or held in a given state in the displayed field of view.

Implementations of the above aspect may include one or more of the following. The imaging device can be an ultrasonic imaging device capable of generating digitized images of the patient target site from any position, respectively, and said tracking device is operable to record the positions of the imaging device at said two positions. The medical instrument can be an endoscope and the view field projected onto the display device is the image seen by the endoscope.

In yet another aspect, machine readable code in a system designed to assist a user in guiding a medical instrument to a target site in a patient, said system including (a) an imaging device for generating one or more intraoperative images, on which a patient target site can be defined in a 3-dimensional coordinate system, (b) a tracking system for tracking the position and optionally, the orientation of the medical instrument and imaging device in a reference coordinate system, (c) an indicator by which a user can indicate a spatial feature of a target site on such image(s), (d) a display device, and (e) an electronic computer operably connected to said tracking system, display device, and indicator, and said code being operable, when used to control the operation of said computer, to (i) record target-site spatial information indicated by the user on said image(s), through the use of said indicator, (ii) determine from the spatial feature of the target site indicated on said image(s), 3-D coordinates of the target-site spatial feature in a reference coordinate system, (iii) track the position of the instrument in the reference coordinate system, (iv) project onto a display device, a view field as seen from a known position and, optionally, a known orientation, with respect to the tool, in the reference coordinate system, and (v) project onto the displayed view field, indicia whose states indicate the indicated spatial feature of the target site with respect to said known position and, optionally, said known orientation, whereby the user, by observing the states of said indicia, can guide the instrument toward the target site by moving the instrument so that said indicia are placed or held in a given state in the displayed field of view.

In yet another aspect, a method for assisting a user in guiding a medical instrument to a subsurface target site in a patient includes indicating a spatial feature of a patient target site on an intraoperative image, determining 3-D coordinates of the patient target site spatial feature in a reference coordinate system using the spatial feature of the target site indicated on the intraoperative image, determining a position of the instrument in the reference coordinate system, projecting onto a display device a view field from a predetermined position relative to the instrument in the reference coordinate system, and projecting onto the view field an indicia of the spatial feature of the target site corresponding to the predetermined position.

Advantages of the system may include one or more of the following. The system enhances intra-operative orientation and exposure in endoscopy, in this way increasing surgical precision and speeding convalescence, which will in turn reduce overall costs. The ultrasound-enhanced endoscopy (USEE) improves localization of targets, such as peri-lumenal lesions, that lie hidden beyond endoscopic views. The system dynamically superimposes directional and targeting information, calculated from intra-operative ultrasonic images, on a single endoscopic view. With USEE, clinicians use the same tools and basic procedures as for current endoscopic operations, but with a higher probability of accurate biopsy, and an increased chance for the complete resection of the abnormality. The system allows for accurate soft-tissue navigation. The system also provides effective calibration and correlation of intra-operative volumetric imaging data with video endoscopy images.

Other advantages may include one or more of the following. The system acquires external 2D or 3D ultrasound images and process them for navigation in near real-time. The system allows dynamic target identification on any reformatted 3D ultrasound cross-sectional plane. The system can automatically track the movement of the target as tissue moves or deforms during the procedure. It can dynamically map the target location onto the endoscopic view in form of a direction vector and display quantifiable data such as distance to target. Optionally, the system can provide targeting information on the dynamic orthographic views (e.g., ultrasound view). The system can also virtually visualize the position and orientation of tracked surgical tools in the orthographic view (e.g., ultrasound view), and optionally also in the perspective (e.g., endoscopic) view.

### BRIEF DESCRIPTION OF THE DRAWINGS

FIGS. 1-2 shows exemplary flow-charts of the operation of one illustrative system.

FIGS. 3-4 shows exemplary operating set-up arrangements and user interface displays in accordance with one aspect of the system.

### DESCRIPTION

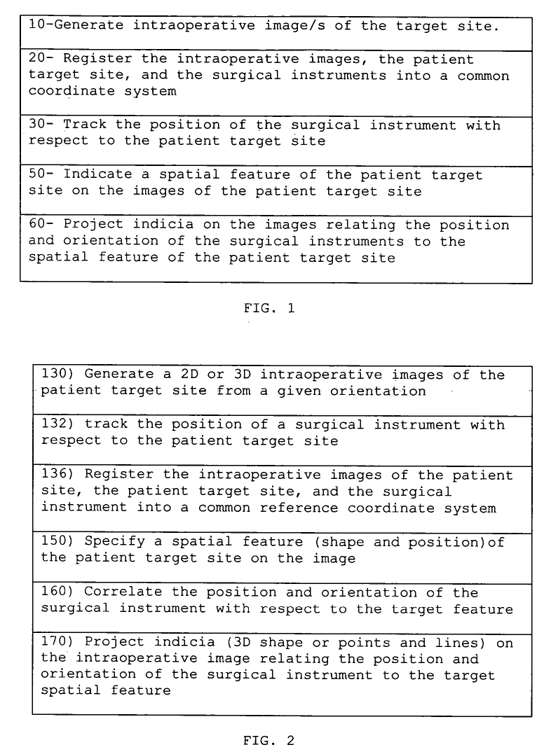

FIG. 1 shows an exemplary process 5 to guide a medical instrument to a desired position in a patient. First, one or more intraoperative images of the target site are acquired (10). Next, the process registers the intraoperative images, the patient target site, and the surgical instruments into a common coordinate system (20). The patient, the imaging source(s) responsible for the intraoperative images and surgical tool must all be placed in the same frame of reference (in registration), and this can be done by one of a variety of methods, among them:
1. Use a tracking device for tracking patient, imaging source(s), and the surgical tool, e.g., a surgical pointer or an endoscope.
2. Track only the position of the tool, and place the tool in registration with the patient and imaging source by touching the tool point to fiducials on the body and to the positions of the imaging source(s). Thereafter, if the patient moves, the device could be registered by tool-to-patient contacts. That is, once the images are made, from known coordinates, it is no longer necessary to further track the position of the image source(s).
3. The patient and image sources are placed in registration by fiducials on the patient and in the images, or alternatively, by placing the imaging device at known coordinates with respect to the patient. The patient and tool are placed in registration by detecting the positions of fiducials with respect to the tool, e.g., by using a detector on the tool for detecting the positions of the patient fiducials. Alternatively, the patient and the surgical tool can be placed in registration by imaging the fiducials in the endoscope, and matching the imaged positions with the position of the endoscope.

Referring back to FIG. 1, the process then tracks the position of the surgical instrument with respect to the patient target site (30). A tracking system is used to track the endoscope for navigation integration in one implementation. In one implementation the system provides a magnetic transducer at the endoscope tip. The tracking system may be calibrated using a calibration jig. A calibration target is modified from a uniform to a non-uniform grid of points by reverse-mapping the perspective transform, so that the calibration target point density is approximately equal throughout the endoscope image.

In one embodiment, an ultrasound calibration system can be used for accurate reconstruction of volumetric ultrasound data. A tracking system is used to measure the position and orientation of a tracking device that will be attached to the ultrasound probe. A spatial calibration of intrinsic and extrinsic parameters of the ultrasound probe is performed. These parameters are used to transform the ultrasound image into the co-ordinate frame of the endoscope's field of view. The calibration of the 3D probe is done in a manner similar to a 2D ultrasound probe calibration. In the typical 2D case, acquired images are subject to scaling in the video generation and capture process. This transformation and the known position of the phantom's tracking device are used to determine the relationship between the ultrasound imaging volume and the ultrasound probe's tracking device. Successful calibration requires an unchanged geometry. A quick-release clamp attached to the phantom will hold the ultrasound probe during the calibration process.

A spatial correlation of the endoscopic video with dynamic ultrasound images is then done. The processing internal to each tracking system, endoscope, and ultrasound machine causes a unique time delay between the real-time input and output of each device. The output data streams are not synchronized and are refreshed at different intervals. In addition, the time taken by the navigation system to acquire and process these outputs is stream-dependant. Consequently, motion due to breathing and other actions can combine with these independent latencies to cause real-time display of dynamic device positions different to those when the imaging is actually being acquired.

A computer is used to perform the spatial correlation. The computer can handle a larger image volume, allowing for increased size of the physical imaged volume or higher image resolution. The computer also provides faster image reconstruction and merging, and a higher-quality rendering at a higher frame rate. The computer time-stamps and buffers the tracking and data streams, then interpolating tracked device position and orientation to match the image data timestamps.

Turning now to FIG. 1, a user indicates a spatial feature of the patient target site on the images of the patient target site (50), and indicia is projected on the images relating the position and orientation of the surgical instruments to the spatial feature of the patient target site (60).

One of the novelties of this system is that it can maintain the registration, mentioned in FIG. 1 (20). The proposed method dynamically tracks and targets lesions in motion beyond the visible endoscopic view. When a target is identified, the subregion surrounding the target in the ultrasound volume will be used to find the new location of the target as it moves during the surgical process. This dynamic tracking will follow each target over time; if the system is displaying target navigation data, the data will change in real time to follow the updated location of the target relative to the endoscope.

Vascular structures return a strong, well differentiated Doppler signal. The dynamic ultrasound data may be rendered in real time making nonvascular structures transparent. This effectively isolates the vascular structure that can be visualized during the navigation process, both in the perspective and orthographic views.

The system of FIG. 1 allows a user such a surgeon to mark a selected target point or region on intraoperative ultrasonic images (one or more ultrasound images). The designated target point or region is then displayed to the surgeon during a surgical operation, to guide the position and orientation of the tool toward the target site. In a first general embodiment, the target area is displayed to the user by displaying a field representing the patient target area, and using the tracked position of the tool with respect to the patient to superimpose on the field, one or more indicia whose position in the displayed field is indicative of the relative position of the tool with respect to the marked target position. In a second general embodiment, the tool is equipped with a laser pointer that directs a laser beam onto the patient to indicate the position and orientation of a trajectory for accessing the target region. The user can follow this trajectory by aligning the tool with the laser-beam.

In the embodiment where the tool is an endoscope, the displayed image is the image seen by the endoscope, and the indicia are displayed on this image. The indicia may indicate target position as the center point of the indicia, e.g., arrows, and tool orientation for reaching the target from that position.

In operation, and with respect to an embodiment using ultrasonic images, the user makes a marking on the image corresponding to the target region or site. This marking may be a point, line or area. From this, and by tracking the position of the tool in the patient coordinate system, the system functions to provide the user with visual information indicating the position of the target identified from the ultrasonic image.

The navigation system operates in three distinct modes. The first is target identification mode. The imaged ultrasound volume will be displayed to allow the surgeon to locate one or more target regions of interest and mark them for targeting. The system can provide navigational information on either 2D plane or three user positionable orthogonal cross-sectional planes for precise 2D location of the target.

In the second mode, the endoscope will be used to set the position and orientation of the frame of reference. Based on these parameters and using the optical characteristics of the endoscope, the system will overlay target navigation data on the endoscope video. This will allow the surgeon to target regions of interest beyond the visual range of the endoscope's field of view. Displayed data will include the directions of, and distances to, the target regions relative to the endoscope tip, as well as a potential range of error in this data.

The third mode will be used to perform the actual interventional procedure (such as biopsy or ablation) once the endoscope is in the correct position. The interactive ultrasound image and cross-sectional planes will be displayed, with the location of the endoscope and the trajectory through its tip projected onto each of the views. The endoscope needle itself will also be visible in the ultrasound displays.

The system allows the interventional tool to be positioned in the center of the lesion without being limited to a single, fixed 2D ultrasound plane emanating from the endoscope tip. In the first implementation of the endoscope tracking system, a magnetic sensor will need to be removed from the working channel in order to perform the biopsy, and the navigation display will use the stored position observed immediately prior to its removal. In another embodiment, a sensor is integrated into the needle assembly, which will be in place at calibration.

The system provides real-time data on the position and orientation of the endoscope, and the ultrasound system provides the dynamic image data. The tip position data is used to calculate the location of the endoscope tip in the image volume, and the probe orientation data will be used to determine the rendering camera position and orientation. Surgeon feedback will be used to improve and refine the navigation system. Procedure durations and outcomes will be compared to those of the conventional biopsy procedure, performed on the phantom without navigation and image-enhanced endoscopy assistance.

The dynamic tracking will follow each target over time; if the system is displaying target navigation data, the data will change in real time to follow the updated location of the target relative to the endoscope.

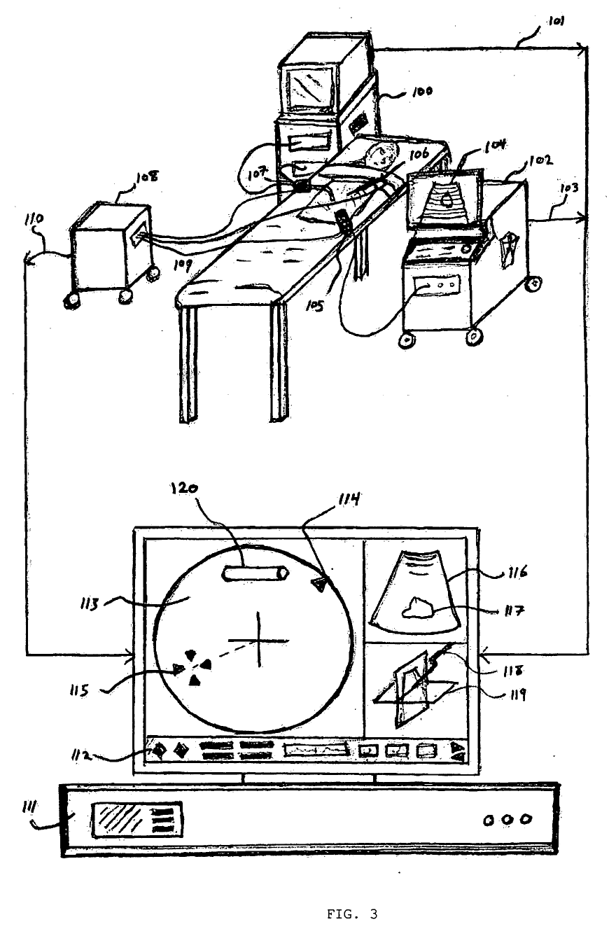

FIG. 2 shows another exemplary implementation where a process acquires one or more 2D or 3D intraoperative images of the patient target site from a given orientation (130). Next, the process tracks the position of a surgical instrument with respect to the patient target site (132). The process then registers the intraoperative images of the patient site, the patient target site, and the surgical instrument into a common reference coordinate system (136). The image of the patient target site and a spatial feature (shape and position) of the patient target site on the image is specified (150). The process then correlates the position and orientation of the surgical instrument with respect to the target feature (160). An indicia (arbitrary shapes or points and lines) is projected on the intraoperative image relating the position and orientation of the surgical instrument to the target spatial feature (170).

Exemplary operating set-up and user interfaces for the systems of FIGS. 1-2 in shown in FIG. 3. In the system of FIG. 3, an endoscopic system 100 or any video source such microscopic or camcorder (not a necessary element anyway) is used to generate a video signal 101. An ultrasonic system 102 (or any intra-operative imaging system) captures an intra-operative imaging data stream 103. The information is displayed on an ultrasonic display 104. A trackable Intra-operative imaging probe 105 is also deployed in one or more trackable surgical tools 106. Other tools include a trackable endoscope 107 or any intraoperative video source. The tracking device 108 has tracking wires 109 that communicate a tracking data stream 110. A navigation system 111 with a navigation interface for ultrasound-enhanced endoscopy 112 is provided to allow the user to work with an intra-operative video image 113 (perspective view) with a superimposed targeting vector 114 and measurement. In the absence of video source this 113 could be blank. Targeting markers 114 (pointing to a target outside the field of view) as well as secondary targeting markers 115 (pointing to a target inside the field of view) can be used. An intra-operative image 116 and an image of the lesion target 117 are shown with a virtual representation of surgical tools or video source 118 (e.g., endoscope) as a reformatted cross sectional planes, called orthographic view 119 (outside view). Additionally, an image overlay 120 of any arbitrary 3D shape (anatomical representation or virtual tool representation) can be shown. The system shown in FIG. 3 can:
work without any intraoperative video source.
track with microscopes and either rigid or flexible endoscopes.
dynamically acquire and process 2D or 3D ultrasound images for navigation.
allow dynamic target identification from the perspective of any given tool.
allow dynamic target identification on any reformatted ultrasound plane.
optionally overlay Doppler ultrasound data, on the video or rendered views.

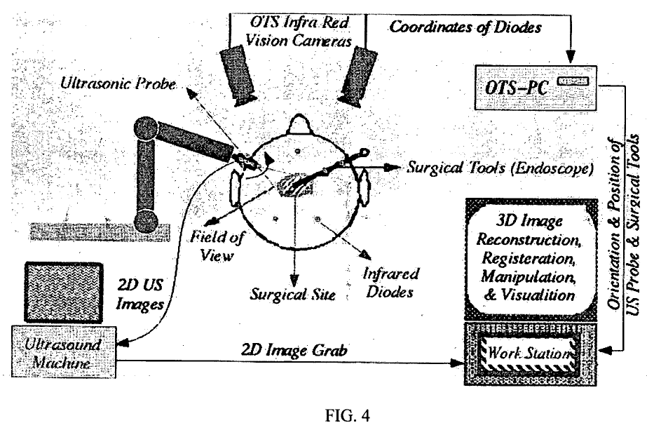

FIG. 4 show another exemplary surgical set-up. In FIG. 4, pluralities of infrared vision cameras track the surgical tools. An ultrasonic probe positions an ultra-sound sensor in the patient. Surgical tools such as an endoscope are then positioned in the patient. The infrared vision cameras report the position of the sensors to a computer, which in turn forwards the collected information to a workstation. The workstation receives data from an ultrasound machine that captures 2D or 3D images of the patient. The workstation also registers, manipulates the data and visualizes the patient data on a screen.

In the event of having to track a flexible endoscope, the field of view at the endoscope tip is not directly dependent on the position of a tracking device attached to some other part of the endoscope. This precludes direct optical or mechanical tracking: while useful and accurate, these systems require an uninhibited line of sight or an obtrusive mechanical linkage, and thus cannot be used when tracking a flexible device within the body.

In order to make use of tracked endoscope video, six extrinsic parameters (position and orientation) and five intrinsic parameters (focal length, optical center co-ordinates, aspect ratio, and lens distortion coefficient) of the imaging system are required to determine the pose of the endoscope tip and its optical characteristics. The values of these parameters for any given configuration are initially unknown.

In order to correctly insert acquired ultrasound images into the volume dataset, the world co-ordinates of each pixel in the image must be determined. This requires precise tracking of the ultrasound probe as well as calibration of the ultrasound image.

One of the advantages of the ultrasound reconstruction engine is that it can be adapted to any existing ultrasound system configuration. In order to exploit this versatility, a simple and reliable tracking-sensor mount capability for a variety of types and sizes of ultrasound probes is used, as it is essential that the tracking sensor and ultrasound probe maintain a fixed position relative to each another after calibration. The surgeon may also wish to use the probe independently of the tracking system and its probe attachment.

Accurate volume reconstruction from ultrasound images requires precise estimation of six extrinsic parameters (position and orientation) and any required intrinsic parameters such as scale. The calibration procedure should be not only accurate but also simple and quick, since it should be performed whenever the tracking sensor is mounted on the ultrasound probe or any of the relevant ultrasound imaging parameters, such as imaging depth or frequency of operation, is modified. An optical tracking system is used to measure the position and orientation of a tracking device that will be attached to the ultrasound probe. In order to make the system practical to use in a clinical environment, spatially calibration of the intrinsic and extrinsic parameters of the ultrasound probe is done. These parameters will then be used to properly transform the ultrasound image into the co-ordinate frame of the endoscope's field of view.

In order to locate and mark the desired region of interest in the ultrasound image, an interface supports interactive rendering of the ultrasound data. An interactive navigation system requires a way for the user to locate and mark target regions of interest. Respiration and other movements will cause the original location of any target to shift. If targets are not dynamically tracked, navigation information will degrade over time.

The imaged ultrasound volume will be displayed to allow the surgeon to locate one or more target regions of interest and mark them for targeting. The system will show an interactive update of the targeting information as well as up to three user positionable orthogonal cross-sectional planes for precise 2D location of the target. In the second mode, the endoscope will be used to set the position and orientation of the frame of reference. Based on these parameters and using the optical characteristics of the endoscope, the system will overlay target navigation data on the endoscope video. This will allow the surgeon to target regions of interest beyond the visual range of the endoscope's field of view. Displayed data will include the directions of, and distances to, the target regions relative to the endoscope tip, as well as a potential range of error in this data. The final mode will be used to perform the actual biopsy once the endoscope is in the correct position. The interactive targeting information and cross-sectional planes will be displayed, with the location of the endoscope and the trajectory through its tip projected onto each of the views. The endoscope needle itself will also be visible in the ultrasound displays.

This will help to position the biopsy needle in the center of the lesion without being limited to a single, fixed 2D ultrasound plane emanating from the endoscope tip, as is currently the case. (That 2D view capability will however be duplicated by optionally aligning a cross-sectional ultrasound plane with the endoscope.) In the first implementation of the flexible endoscope tracking system, the tracking sensor will need to be removed from the working channel in order to perform the biopsy, and the navigation display will use the stored position observed immediately prior to its removal. Ultimately, though, a sensor will be integrated into the needle assembly, which will be in place at calibration.

This dynamic tracking will follow each target over time; if the system is displaying target navigation data, the data will change in real time to follow the updated location of the target relative to the endoscope.

Lens distortion compensation is performed for the data display in real time, so that the superimposed navigation display maps accurately to the underlying endoscope video.

A new ultrasound image will replace the next most recent image in its entirety, much as it does on the display of the ultrasound machine itself, although possibly at a different spatial location. This avoids many problematic areas such as misleading old data, data expiration, unbounded imaging volumes, and locking rendering data. Instead, a simple ping-pong buffer pair may be used; one may be used for navigation and display while the other is being updated. Another benefit of this approach is that the reduced computational complexity contributes to better interactive performance and a smaller memory footprint.

The invention has been described in terms of specific examples which are illustrative only and are not to be construed as limiting. The invention may be implemented in digital electronic circuitry or in computer hardware, firmware, software, or in combinations of them. Apparatus of the invention may be implemented in a computer program product tangibly embodied in a machine-readable storage device for execution by a computer processor; and method steps of the invention may be performed by a computer processor executing a program to perform functions of the invention by operating on input data and generating output. Suitable processors include, by way of example, both general and special purpose microprocessors. Storage devices suitable for tangibly embodying computer program instructions include all forms of non-volatile memory including, but not limited to: semiconductor memory devices such as EPROM, EEPROM, and flash devices; magnetic disks (fixed, floppy, and removable); other magnetic media such as tape; optical media such as CD-ROM disks; and magneto-optic devices. Any of the foregoing may be supplemented by, or incorporated in, specially-designed application-specific integrated circuits (ASICs) or suitably programmed field programmable gate arrays (FPGAs).

From the a foregoing disclosure and certain variations and modifications already disclosed therein for purposes of illustration, it will be evident to one skilled in the relevant art that the present inventive concept can be embodied in forms different from those described and it will be understood that the invention is intended to extend to such further variations. While the preferred forms of the invention have been shown in the drawings and described herein, the invention should not be construed as limited to the specific forms shown and described since variations of the preferred forms will be apparent to those skilled in the art. Thus the scope of the invention is defined by the following claims and their equivalents.

1. A method for guiding a medical instrument to a target site within a patient, comprising:
capturing at least one intraoperative image from the patient;
identifying a spatial feature indication of a patient target site on the intraoperative image,
determining coordinates of the patient target site spatial feature in a reference coordinate system,
determining a position of the instrument in the reference coordinate system,
creating a view field from a predetermined position, and optionally orientation, relative to the instrument in the reference coordinate system, and
projecting onto the view field an indicia of the spatial feature of the target site corresponding to the predetermined position, and optionally orientation.

2. The method of claim 1, further comprising
a. using an ultrasonic source to generate an ultrasonic image of the patient, and
b. determining coordinates of a spatial feature indicated on said image from the coordinates of the spatial feature on the image and the position, and optionally orientation, of the ultrasonic source.

3. The method of claim 1, wherein said medical instrument is a source of video and the view field projected onto the display device is the image seen by the video source.

4. The method of claim 1, wherein the view field projected onto the display device is that seen from the tip-end position and orientation of the medical instrument having a defined field of view.

5. The method of claim 1, wherein the view field projected onto the display device seen from a position along the axis of instrument different from the target seen at a tip-end position of the medical instrument.

6. The method of claim 1, wherein the target site spatial feature indicated is a volume or an area, and said indicia are arranged in a geometric pattern defining a boundary of the indicated spatial feature.

7. The method of claim 1, wherein the target site spatial feature indicated is a volume, an area or a point, and said indicia are arranged in a geometric pattern indicating a position of a point within the target site.

8. The method of claim 1, wherein a spacing between or among indicia is indicative of the distance of the instrument from the target-site position.

9. The method of claim 1, wherein the size or shape of the individual indicia is indicative of the distance of the instrument from the target-site position.

10. The method of claim 1, wherein the size or shape of individual indicia is indicative of the orientation of said instrument.

11. The method of claim 1, wherein said indicating includes indicating on each image, a second spatial feature which, together with the first-indicated spatial feature, defines a surgical trajectory on the displayed image.

12. The method of claim 1, which further includes using said instrument to indicate on a patient surface region, an entry point that defines, with said indicated spatial feature, a surgical trajectory on the displayed image.

13. The method of claim 11 or 12, wherein the surgical trajectory on the displayed image is indicated by two sets of indicia, one set corresponding to the first-indicated spatial feature and the second set by the second spatial feature.

14. The method of claim 11 or 12, wherein the surgical trajectory on the displayed image is indicated by a geometric object defined, at its end regions, by the first-indicated spatial feature and the second spatial feature or entry point indicated.

15. The method of claim 1, further comprising moving the instrument toward the target site by moving the instrument so that said indicia are placed or held in a given state in the displayed field of view.

16. A system designed to a user in guiding a medical instrument to a target site in a patient, comprising
(a) an imaging device for generating one or more intraoperative images with spatial features of a patient target site in a 3-dimensional coordinate system;
(b) a tracking system for tracking the position and optionally, the orientation of the medical instrument and imaging device in a reference coordinate system,
(c) an indicator by which a user can indicate a spatial feature of a target site on the one or more images,
(d) an electronic computer operably connected to said tracking system, display device, and indicator, and
(e) computer-readable code which is operable, when used to control the operation of the computer, to carry out the steps of
(i) recording target-site spatial information indicated by the user on said image(s), through the use of said indicator,
(ii) determining from the spatial feature of the target site indicated on said image(s), 3-D coordinates of the target-site spatial feature in a reference coordinate system,
(iii) tracking the position of the instrument in the reference coordinate system,
(iv) creating a view field as seen from a known position and, optionally, a known orientation, with respect to the tool, in the reference coordinate system, and
(v) projecting onto the view field, indicia whose states indicate the indicated spatial feature of the target site with respect to said known position and, optionally, said known orientation,

17. Machine readable code in a system designed to assist a user in guiding a medical instrument to a target site in a patient, said system including
(a) an imaging device for generating one or more intraoperative images, on which a patient target site can be defined in a 3-dimensional coordinate system,
(b) a tracking system for tracking the position and optionally, the orientation of the medical instrument and imaging device in a reference coordinate system,
(c) an indicator by which a user can indicate a spatial feature of a target site on such image(s),
(d) an electronic computer operably connected to said tracking system, display device, and indicator, said code being operable, when used to control the operation of said computer, to:
(i) record target-site spatial information indicated by the user on said image(s), through the use of said indicator,
(ii) determine from the spatial feature of the target site indicated on said image(s), 3-D coordinates of the target-site spatial feature in a reference coordinate system,
(iii) track the position of the instrument in the reference coordinate system,
(iv) create a view field as seen from a known position and, optionally, a known orientation, with respect to the tool, in the reference coordinate system, and
(v) project onto the view field, indicia whose states indicate the indicated spatial feature of the target site with respect to said known position and, optionally, said known orientation,

18. The code of claim 18, further comprising code to allow a user, by observing the states of said indicia, to guide the instrument toward the target site by moving the instrument so that said indicia are placed or held in a given state in the displayed field of view.

19. The code of claim 18, wherein the target site spatial feature indicated is a volume or an area, and said indicia are arranged in a geometric pattern defining a boundary of the indicated spatial feature.
